(19) United States
(12) Patent Application Publication
(10) Pub. No.: US 2010/0240768 A1
(43) Pub. Date: Sep. 23, 2010

(54) NOVEL NUTRACEUTICALCOMPOSITIONS CONTAINING THYMOL AND/OR P-CYMENE OR PLANT EXTRACTS FOR COGNITION

(76) Inventors: Ann Fowler, Rheinfelden (CH); Regina Goralczyk, Grenzach-Wyhlen (DE); Claus Kilpert, Mannheim (DE); Annis Mayne-Mechan, Moehlin (CH); Hasan Mohajeri, Egg. b. Zuerich (CH); Bernd Mussler, Lahr (DE); Adrian Wyss, Basel (CH)

Correspondence Address: NIXON & VANDERHYE, PC, 901 NORTH GLEBE ROAD, 11TH FLOOR, ARLINGTON, VA, 22203, US

(21) Appl. No.: 12/738,729

(22) PCT Filed: Oct. 17, 2008
(86) PCT No.: PCT/EP08/08821

§ 371 Date: Apr. 19, 2010

(30) Foreign Application Priority Data
Oct. 18, 2007 (EP) 07020345.0
Aug. 8, 2008 (EP) 08014178.1

(51) Int. Cl. A61K 31/05; A61K 31/015; A61P 25/00

(52) U.S. Cl. 514/731; 514/764

(57) ABSTRACT

The invention relates to a novel nutraceutical composition containing thymol and/or p-cymene, or a plant extract containing thymol or p-cymene as active ingredient(s). The compositions are useful for improvement of cognitive functions and psycho-social status, such as learning, memory and alertness, psychotic stability and maintenance.

### FIELD OF THE INVENTION

The present invention relates to a novel nutraceutical composition or food additive comprising thymol and/or p-cymene or a plant extract comprising thymol and/or p-cymene as active ingredient(s) to improve cognitive functions such as learning, memory and alertness as well as relieving psychosocial pressure. Thymol, p-cymene and enriched thyme extracts are also useful for treating conditions resulting from hypoxia, for alleviating neuropathic pain and psychotic conditions.

### BACKGROUND OF THE INVENTION

Memory, learning and alertness rely on neuronal circuits in the midbrain, especially in the hippocampus where information is processed and memory is consolidated. Mental performance and learning are dependent on synaptic plasticity; i.e. strengthening neuronal connections by the recruitment of new receptors, formation of new synapses and eventually the generation of new neuronal connections.

The formation of (long-term) memory and the efficient functioning of the brain depend on synthesis of new proteins for the reinforcement of communicative strength between neurons. The production of new proteins devoted to synapse reinforcement is triggered by chemical and electrical signals within neurons.

Long term potentiation (LTP) is the term used to describe the long-lasting enhancement of synaptic transmission (hours in vitro, days or weeks in vivo) which occurs at particular synapses within the central nervous system (CNS) following a short, conditioning, burst of presynaptic electrical stimulation (approximately 100 Hz for 1 second). This phenomenon is widely considered to be one of the major mechanisms by which memories are formed and stored in the brain. LTP has been observed both in vitro and in living animals. Under experimental conditions, applying a series of short, high-frequency electric stimuli to a synapse can potentiate the strength of the chemical synapse for minutes to hours. Most importantly, LTP contributes to synaptic plasticity in living animals, providing the foundation for a highly adaptable nervous system.

Two different receptor types are primarily involved in the process of LTP, namely the N-methyl-D-aspartate (NMDA) receptor complex and the α-amino-3-hydroxy-5-methyl-4-isoxazole propionic acid (AMPA) receptor. During LTP, the major excitatory neurotransmitter, glutamate, is released from the presynaptic neuron, binds to and activates the AMPA receptor on the postsynaptic membrane, leading to its depolarisation. At resting membrane potentials, the NMDA receptor channel is blocked by magnesium ions, but depolarisation of the postsynaptic membrane removes this block, enabling NMDA receptor activation and subsequent entry of calcium into the cell. This rise in intracellular calcium is believed to activate protein kinases, leading to gene transcription and the construction of reinforcing proteins (Neihoff 2005, Speak Memory 210-223) and resulting in enhanced sensitivity of the AMPA receptor, thus further facilitating neurotransmission and maintenance of LTP.

NMDA receptors are composed of assemblies of NR1- and NR2-subunits; the glutamate binding domain is formed at the junction of these subunits. In addition to glutamate, the NMDA receptor requires a co-agonist, glycine, in order to modulate receptor function. The glycine binding site is found on the NR1 subunit, while the NR2 subunit possesses a binding site for polyamines, regulatory molecules that modulate the functioning of the NMDA receptor.

The amino acid glycine is thus known to act as a positive allosteric modulator and obligatory co-agonist with glutamate at the NMDA receptor complex (Danysz 1998, Pharmacol. Rev., 50 (4), 597-664). Glycine transporters (GlyT) play an important role in the termination of postsynaptic glycinergic actions and maintenance of low extracellular glycine concentrations by reuptake of glycine into presynaptic nerve terminals or glial cells. The termination of the action of glycine is therefore largely mediated by rapid reuptake. Two glycine transporters, GlyT1 and GlyT2, are known and are characterized by 12 putative transmembrane regions, while three variants of GlyT 1 (GlyT1a, b, and c) encoded from the same gene have been identified (Borowsky and Hoffman (1998), J. Biol. Chem., 273 (44), 29077-29085).

GlyT1 is the only sodium chloride-dependent glycine transporter in the forebrain, where it is co-expressed with the NMDA receptor. At this site, GlyT1 is thought to be responsible for controlling extracellular levels of glycine at the synapse (López-Corcuera (2001), Mol. Membr. Biol., 18 (1), 13-20), resulting in modulation of NMDA receptor function.

Indeed, in the presence of the selective GlyT1 antagonist N-[3-(4′-fluorophenyl)-3-(4′-phenylphenoxy)]propylsarcosine (NFPS), enhanced NMDA receptor responses in CA1 pyramidal cells were observed upon Schaffer collateral stimulation in both mouse and rat hippocampal slice preparations (Bergeron, et al (1998), Proc. Natl. Acad. Sci. USA, 95 (26), 15730-15734). In vivo, systemic administration of NFPS increased LTP in the dentate gyrus and enhanced prepulse inhibition of the acoustic startle response in adult mice, indicating that inhibition of GlyT1 affects NMDA receptor function in a behaviourally-relevant way (Kinney, et al (2003), J. Neurosci., 23 (20), 7586-7591).

Such data highlight the potential usefulness of compounds which can enhance NMDA receptor synaptic function by elevating extracellular levels of glycine in the local microenvironment of synaptic NMDA receptors, for the prevention of psychotic syndromes and for maintaining or boosting physiological cognitive functions, such as memory and learning, in normal individuals.

There is an increasing interest in the development of compounds, as well as nutraceutical compositions, that may be used to improve learning, memory and alertness, in both elderly and young people, individuals who need especially high memory and attention in their daily work, including students, construction workers, drivers, pilots, physicians, salespeople, executives, housewives, “high performance professionals” and people who are under mental or daily stress as well as persons who are prone to psychiatric instability, such as schizophrenia.

Thus, a compound or nutraceutical composition which enhances NMDA receptor function and enables improvements in learning, memory and alertness would be highly desirable.

JP2004002237 discloses the use of an anti-aging foodstuff or pharmaceutical, which includes rosemary as one of many plant sources of anti-aging compounds. One of the claimed uses of this composition is the improvement of learning function and memory, in addition to beneficial effects on hair and skin and eye- and bone-health.

### DETAILED DESCRIPTION OF THE INVENTION

It has been found, in accordance with this invention that compounds of Formula 1, below, or a salt, derivative, metabolite or analogue thereof, are activators of hippocampal function, through their ability to induce LTP. They can work either by inhibiting the glycine transporter, GlyT1, thus inhibiting reuptake of glycine, or by activation of another pathway, or by both mechanisms. These biological activities are important in memory formation and memory consolidation, thus these compounds are useful in enhancing cognitive functions.

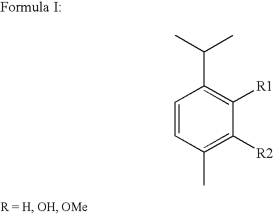

### Wherein R1=H, OH or OMe; and

### R2=H, OH, or OMe

In particular, it has been found in accordance with the invention, that thymol, and plant extracts which contain thymol, have the ability to inhibit glycine reuptake by inhibiting the glycine transporter, GlyT1. The resulting increase in extracellular glycine levels leads to additional activation of NMDA receptors, which is the first step to inducing transcriptional activation of a number of genes and subsequently to induce LTP, the main cellular mechanism involved in memory formation and memory consolidation.

It has also been found that p-cymene, another compound which can be found in plant extracts, enables induction of LTP through a different mechanism. As both processes have the same biological benefits, i.e. they both facilitate hippocampal functioning leading to enhanced cognitive functioning, another aspect of this invention is the use of these two active ingredients together to enhance cognitive functions.

Therefore one aspect of the invention is a novel nutraceutical composition, comprising a compound of Formula I or a thymoacetate to enhance cognitive functions. Particularly preferred compounds of Formula I are thymol and p-cymene.

The compounds of Formula I can either be synthetically produced using known methods; they can be extracted from natural sources such as plants using known extraction procedures, or they may be used as a component of a plant extract, preferably a thyme extract which contains sufficient amounts of thymol and/or p-cymene to be an effective enhancer of hippocampal function.

### BRIEF DESCRIPTION OF THE FIGURES

FIG. 1 shows dose-response curves of thymol and enriched thyme extracts in the GlyT1 inhibition assay. Assay results are presented as the % inhibition of internalization of radioactive glycine into the cells. FIG. 1 clearly demonstrates that two different thyme extracts as well as the most prominent volatile component of thyme, thymol, can specifically inhibit the action of GlyT1 in a cellular assay.

FIG. 1a shows that for ALX, the IC₅₀=6.46 nM

FIG. 1b shows that for Sarcosine, the IC₅₀=35.9 nM

FIG. 1c shows that for ORG 24569, the IC₅₀=0.02 μM

FIG. 1d shows that for Thymol, the IC₅₀=13.6 μM

FIG. 1e shows that for Thyme extract 1, the IC₅₀=48.2 μg/mL

FIG. 1f shows that for Thyme extract 2, the IC₅₀=45.1 μg/mL

FIG. 2a shows the results from the step-down behavioral testing, expressed as number of errors. Mice treated with thyme extract performed significantly better than their age-matched controls and comparable to the mice treated with positive controls. No statistical difference was observed between the performance of the thyme-treated groups and the ginkgo or rolipram-treated groups at any time. a: significant difference to vehicle treated age-matched littermates during the training period. b: significant difference to vehicle treated age-matched littermates during the test period. c: significant difference to vehicle treated age-matched littermates during the washout period.

FIG. 2b. Step-down behavioral testing, duration of latency. Mice treated with thyme extract performed significantly better than their age-matched controls and comparable to the mice treated with positive controls. No statistical difference was observed between the performance of the thyme-treated groups and the ginkgo or rolipram-treated groups at any time.

FIG. 3a: Visit duration in each corner 3 h before and after objects were presented. The filled circles represent the place of an object.

FIG. 3b: Visiting times in each corner were normalized to total time spent in all 4 corners before the presentations of the objects.

FIG. 3c: Learning curve during the active phase for thyme extract treated group in comparison with control and Ginkgo biloba (GBE) treated animals. All groups performed better over time, although no difference among the control and treatment groups could be found (p=0.44). This data show that all animals could learn the task.

FIG. 3d: Reversal of place learning: Errors were recorded during the first active phase. Error rates drop from 100% to about 20% for thyme extract and about 60-70% for both control groups (p=0.011 for the last two time bins). Time bins correspond to 2 h each.

### PLANT EXTRACTS

There are a number of plant species which contain thymol and/or p-cymene and may be the source of the plant extract. Preferably the plant is a member of the genus Thymus, as it contains both compounds, but the source of the extract may be any plant known to contain either compound. Examples of other plants known to contain thymol/p-cymene include: Horsemint (Monarda punctata and related Monarda species such as M. fistulosa), Ajowan caraway (Trachyspermum ammi), dill (Anethum graveolens), fenugreek (Trigonella foenum-graecum), winter savory (Satureja montana), celery (Apium graveolens), tea tree (Melaleuca alternifolia) and true cardamom (Elettaria cardamomum).

The thyme extract may be made from any species of the genus Thymus, such as T. vulgaris, T. zygis, T. pulegioides, T. serpyllum, T. bournmuelleri, T. decassatus, T. longicaulus, T. syriacus and Thymus schimp. Preferably the Thymus is Thymus vulgaris. Generally, the thyme extract should contain at least about 25-80% thymol, preferably from about 40-65% thymol, and more preferably from about 50-60% thymol. Generally the thyme extract should contain at least about 5-55% p-cymene, and preferably from about 10-40% p-cymene.

As used throughout the specification and claims, the term “thyme extract” is intended to be used broadly, and can encompass plant extracts made by conventional means, such as steam distillation, supercritical CO₂(SF—CO₂) extractions, water-based extractions, nitrous oxide extractions, alcohol-based extractions, or organic solvent-based extractions, such as ethyl acetate, propane, acetone, optionally modified with modifiers such as ethanol. As the extract is intended for human and animal consumption, the extract should be one which is approved by regulatory agencies.

The only critical parameters regarding the thyme extract are:
1) It should be acceptable for use in a nutraceutical composition for animal or human consumption. Thus the solvent to be employed for its preparation should be approved by the various regulatory agencies for the intended use. Therefore, preferred extraction procedures are steam distillation, SF—CO₂, C₂₋₄alcohol extractions and ethyl acetate extractions.
2) It should contain a sufficient amount of the active compounds thymol, or p-cymene, or both so that the desired effects, such as cognition improvement and/or improvement in psycho-social status are achieved, i.e. the subject exhibits an improved memory function or can cope with stressful situations in a more productive fashion. As used throughout the specification and claims, the terms “cognitive function-improving amount” and “psycho-social status-improving amount” are intended to convey this concept. The amount of improvement of the various states can be assessed using standardized psychological assessment tests.

As used throughout the specification and claims, the term “extract” includes conventional extracts (i.e. a total extract, such as a standard lipophilic extract) as well as those extracts which have been produced using two or more extraction procedures (“enriched” extracts, where the total extract has been further refined, often by using a second extraction, in order to concentrate desired constituents.)

Thyme extracts typically contain other compounds which may also be bioactive, and/or increase the bioavailability of the active components, thymol and/or p-cymene. The amounts in which they are present in the thyme extract will vary, based on a number of factors, including: the species of Thymus used, the growing conditions of the plant, and, of course, the processes used to prepare the thyme extract. A typical Thymus vulgaris extract prepared using supercritical CO₂ methods will contain (in addition to thymol and p-cymene): carvacrol, 1,8-cineol, borneol, geraniol, linalool, bornyl, linalyl acetate, thymol methyl ether and a-pinene, apigenin, luteolin, thymonin, naringenin and caryophyllene.

### Thymol Benefits Mental States

As previously described, the basis of memory, learning and mental stability is LTP, or the strengthening of neuronal connections, which occurs via activation of AMPA and NMDA receptors within the brain, particularly in the hippocampus.

As glycine reuptake inhibitors through their activity at GlyT1, thymol, and thyme extracts containing thymol, enable accumulation of glycine in the vicinity of the NMDA receptor, thus activating it and ultimately resulting in the induction of LTP, the main cellular mechanism involved in memory formation and memory consolidation.

Moreover, p-cymene induces activation of the same biochemical pathway (albeit at a different step than thymol), leading to LTP induction, and is likewise beneficial in improving memory functions. Thus, thymol and p-cymene, and extracts containing either or both, can activate hippocampal functions and improve memory formation and consolidation, as well as improve mental health.

Conditions Improved by this Invention:

In the context of this invention “treatment” also encompasses co-treatment as well as prevention. Prevention can mean lessening the risk of development of a condition, ameliorating a condition, early intervention, and or minimizing the severity of a condition which develops in a future time.

Throughout this specification and claims, the term “improved cognitive function” is meant to refer to the conditions of supporting and maintaining cognitive wellness and balance, such as:
Enhanced learning, including:
   language processing
   problem solving
   intellectual functioning
Ability to cope with psychosocial burdens
Enhanced attention and concentration
Enhanced memory and the capacity for remembering, especially short-term memory
Enhanced mental alertness and mental vigilance, reduction of mental fatigue
Stabilization of mental status including:
   Relieving post-partum conditions
   Relieving psychological burden due to separation of partners, children, death of beloved people or marital problems
   Relieving problems associated with change of domicile, work or similar events
   Relieving stressful conditions following a traffic accident or other negative social pressure
Stress relief, including:
   treatment, prevention and alleviation of symptoms related to work overload, exhaustion and/or “burn out”
   increased resistance or tolerance to stress
   favouring and facilitating relaxation in normal healthy individuals
“Condition improvement”, including:
   reducing irritability and tiredness
   reducing, preventing or alleviating physical and mental fatigue
   promoting good-quality sleep, that is to act against insomnia and sleep disorders and to increase energy in more general terms, in diseased or normal healthy individuals.

In a preferred aspect of the present invention the compositions may be used as nutritional supplements, particularly for people who may feel a special need for enhanced cognitive function and/or psychosocial support. A non-exhaustive list of people who would benefit from enhanced cognitive function would include:
elderly people,
students or persons who are preparing for exams,
children who are engaged in a great deal of learning, i.e. infants, toddlers, pre-school children and school children,
construction workers, or those operating potentially dangerous machinery,
truck drivers, pilots, train drivers, or other transportation professionals,
air traffic controllers,
salespeople, executives, and other “high performance professionals”
police officers and military personnel,
housewives,
   or for anyone exposed to high amounts of stress in their daily work or who needs especially high attention/concentration/high mental and psychological performance in their daily work, such as those participating in sports, chess players, golfers, professional performers (actors, musicians and the like).

To achieve these improvements, administration over several days (for example at least 6 or 10 days) is recommended, and administration daily for several weeks is generally preferred.

Aside from applications for humans, the compositions of this invention have additional uses in the veterinary world. Animals which can benefit from enhanced cognitive function include those animals which are subject to stressful conditions. Such conditions occur, for example, after capture or transport or may be due to housing conditions (such as change of domicile or owner), when the animals develop analogous disorders and are distressed or aggressive, or display stereotypic behavior, or anxiety and obsessive-compulsive behavior. Animals which are subject to stress would also include those which are racing animals (e.g. dogs, horses, camels), or used in various sports, performing animals (such as circus animals and those appearing on stage, television or in the movies) and horses which perform dressage and other highly disciplined routines.

Preferred “animals” are pets or companion animals and farm animals. Examples of pets are dogs, cats, birds, aquarium fish, guinea pigs, (jack) rabbits, hares and ferrets. Examples of farm animals are aquaculture fish, pigs, horses, ruminants (cattle, sheep and goats) and poultry.

### Nutraceutical Uses/Formulations/Dosages

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form that is suitable for administering to the body, especially in any form that is conventional for oral administration, e.g. in solid forms such as (additives/supplements for) food or feed, food or feed premix, fortified food or feed, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Examples of food are dairy products including, for example, margarines, spreads, butter, cheese, yoghurts or milk-drinks.

Examples of fortified food are cereal bars, bakery items, such as cakes and cookies, and potato chips or crisps.

Beverages encompass non-alcoholic and alcoholic drinks as well as liquid preparations to be added to drinking water and liquid food. Non-alcoholic drinks are e.g. soft drinks, sports drinks, fruit juices, lemonades, teas and milk-based drinks. Liquid foods are e.g. soups and dairy products. The nutraceutical composition containing thymol and/or an enriched thyme extract or p-cymene may be added to a soft drink, an energy bar, or a candy, such that an adult consumes a cognitive-function improving amount of thymol or thyme-containing plant extract, ranging from about 10 to 1000 mg per daily serving, preferably from about 50 to 750 mg per daily serving, or more preferably from about 100 to 500 mg per daily serving. For p-cymene, a cognitive-function improving amount ranges from about 5 to 500 mg per daily serving, preferably from about 25 to 375 mg per daily serving and more preferably from about 50 to 250 mg per daily serving.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives. Daily dosages are substantially the same as those above for food formulations, but for ease of administration, may be divided into smaller doses per administration unit, and multiple administration units (such as 1-4 capsules) may be taken daily. Preferred are thymol and/or enriched thyme-containing plant extract derivates which, when taken together provide a daily dosage of 10-1000 mg, more preferably 50-750 mg, and even more preferably 100-500 mg. For p-cymene, the preferred daily dosage is about 5-500 mg, preferably 25-375 mg, and even more preferably 50-250 mg.

For animals excluding humans a suitable daily dosage of thyme or a thyme extract, for the purposes of the present invention may be within the range from 0.001 mg per kg body weight to about 1000 mg per kg body weight per day. More preferred is a daily dosage of about 0.1 mg to about 500 mg per kg body weight, and especially preferred is a daily dosage of about 1 mg to 100 mg per kg body weight.

The following non-limiting Examples are presented to better illustrate the invention.

### EXAMPLE 1

### Preparation and Composition of Thyme Extract

Commercial suppliers of suitable thyme extracts include MDidea (MDidea Exporting Division, No. 9, WBSS, Ntez. YC, China), FLAVEX (FLAVEX Naturextrakte GmbH, Nordstrasse 7, D-66780 Rehlingen, Germany) and White Lotus Aromatics (602 S. Alder Street, Port Angeles, Wash. 98362-6612, USA).

### Preparation and Composition of Thyme Extracts

In the Examples below, “-se” refers to phenol-type thyme Extract 1, and “-to” refers to terpineol-type thyme Extract 2, both obtained from Flavex, Germany.

Dried leaves of thyme were milled and extracted with supercritical carbon dioxide. The parameters of extraction were as follows: temperature of 45° C.; working pressure: 300 bar (-to) or 100 bar (-se); 17 kg (-to) and 15 kg (-se) of carbon dioxide per 1 kg of plant material were needed; the extracts were obtained in the separator by throttling the pressure to 60 bar at 30° C. 25 kg (-to) or 50 kg (-se) of plant material respectively yielded 1 kg of extract.

Extract 1 (-se) had the following composition (analysed by Gas Chromatography):

Total content of essential oil was 65.3% (the remaining parts are plant waxes).

Volatile components are listed below:

| | |
|---|---|
| Thymol | 53% |
| P-Cymene | 34% |
| Linalool | 2.2% |
| Caryophyllene | 2% |
| Carvacrol | 1.7% |

Extract 2: (-to) contained 47.8% essential oils. The composition of volatile compounds is listed below:

| | |
|---|---|
| Thymol | 52.0% |
| p-Cymene | 18.7% |
| gamma Terpinene | 7.2% |
| Carvacrol | 3.7% |
| Caryophyllene | 3.7% |
| Linalool | 3.7% |
| Borneol | 1.5% |
| beta Myrcene | 1.2% |
| 1,8 Cineol | 1.1% |
| alpha Pinene | 0.6% |
| Limonene | 0.3% |

### EXAMPLE 2

### Inhibition of Glycine Transporter 1 in a Cellular Assay

CHO cells stably expressing the human glycine transporter 1b cDNA (GlyT1) were routinely grown in Dulbecco's Modified Eagle's Medium (purchased from Invitrogen, Carlsbad, USA) containing 10% dialyzed fetal calf serum, penicillin, streptomycin, proline and the antibiotic G418. Cells were harvested by trypsinisation one day prior to the assay and were seeded in the above mentioned medium. Immediately prior to the assay, the medium was replaced by uptake buffer containing 150 mM NaCl, 1 mM CaCl₂, 2.5 mM KCl, 2.5 mM MgCl₂, 10 mM Glucose and 10 mM N-2-hydroxyethylpiperazine-N′-2-ethanesulfonic acid (“Hepes” buffer).

Glycine uptake into the cells was determined by addition of 60 nM radio-labelled [³H] glycine (Amersham Biosciences GE Healthcare, Slough, UK) and incubation for 30 minutes at room temperature. Following removal of unincorporated label by gentle washing three times with the above buffer, incorporated glycine was quantified by liquid scintillation counting.

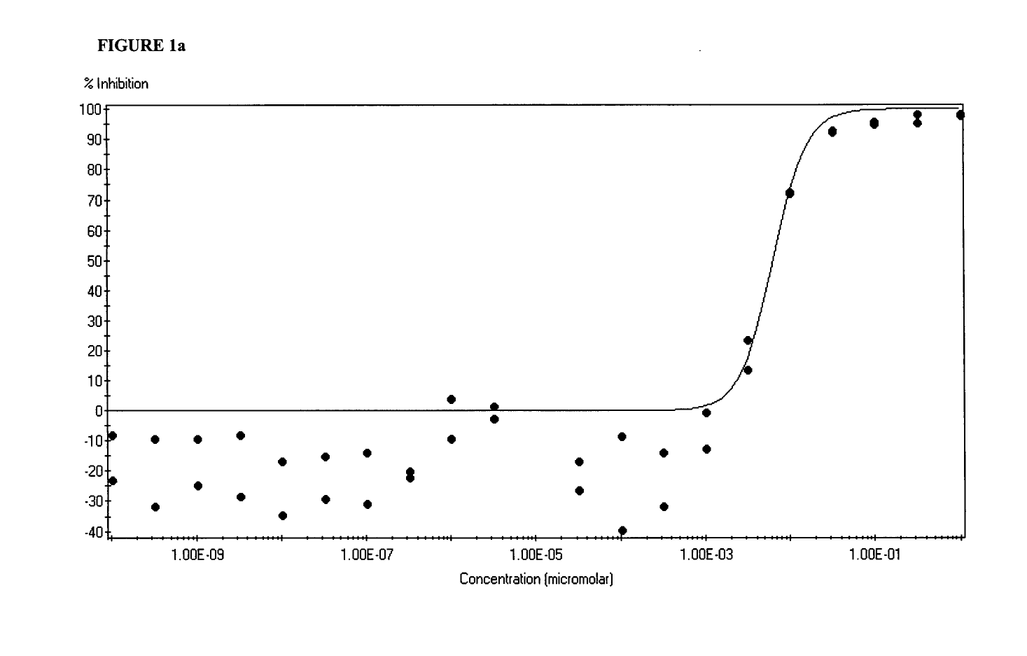

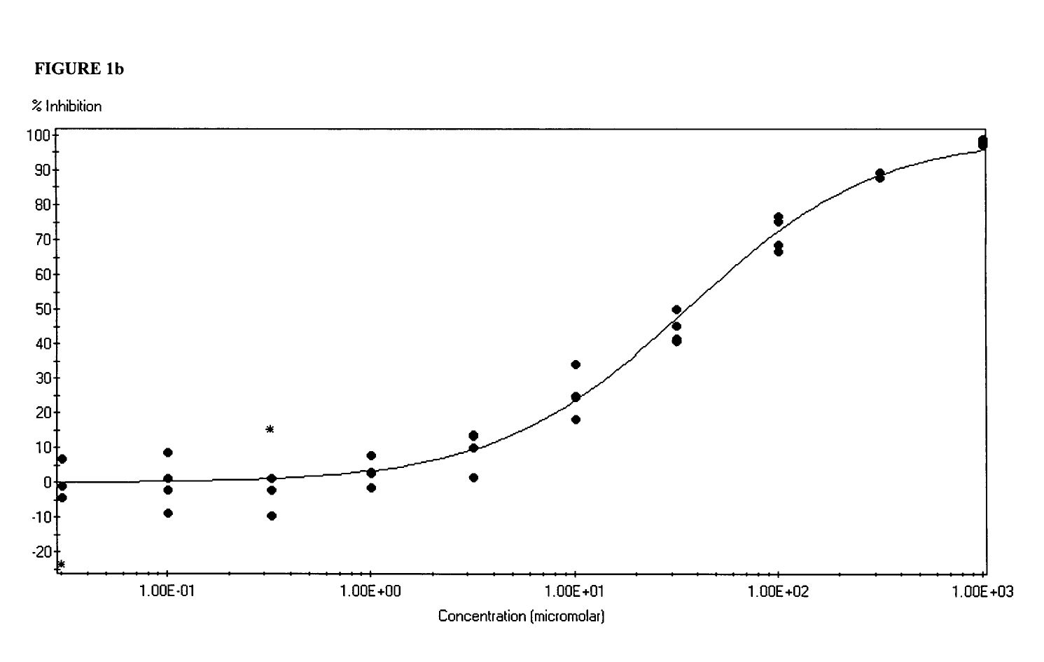

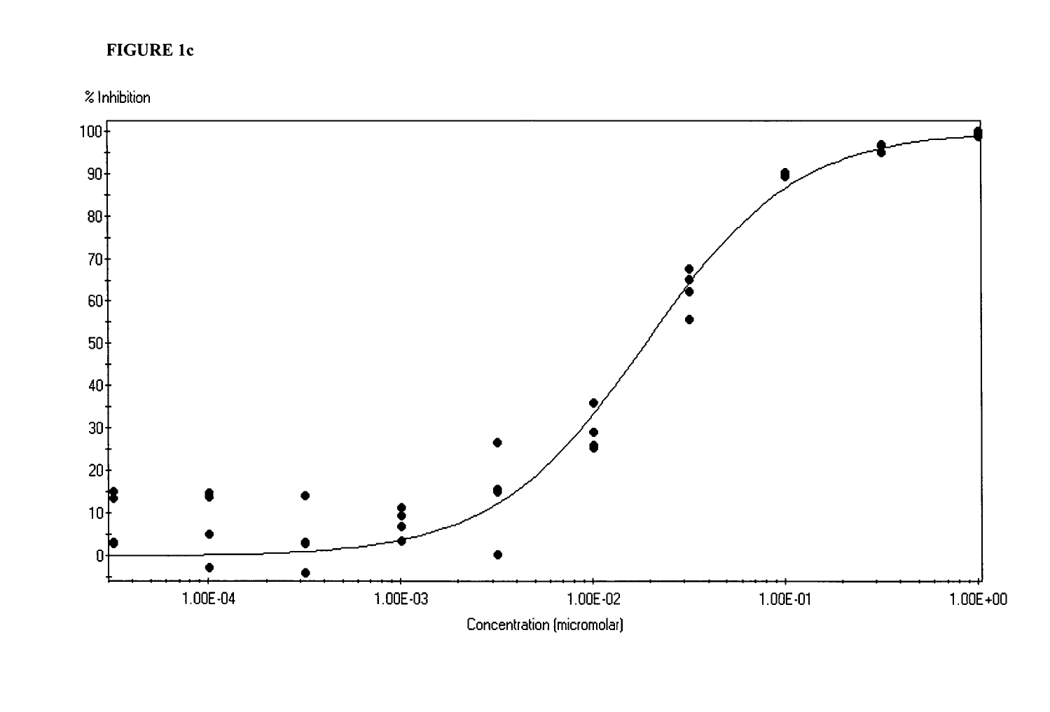

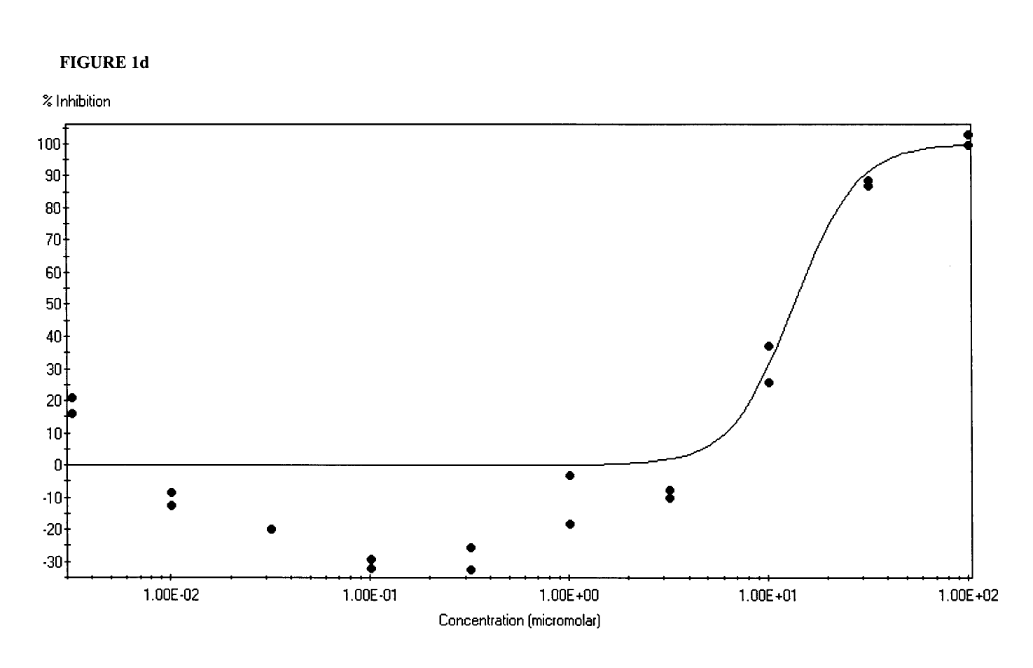

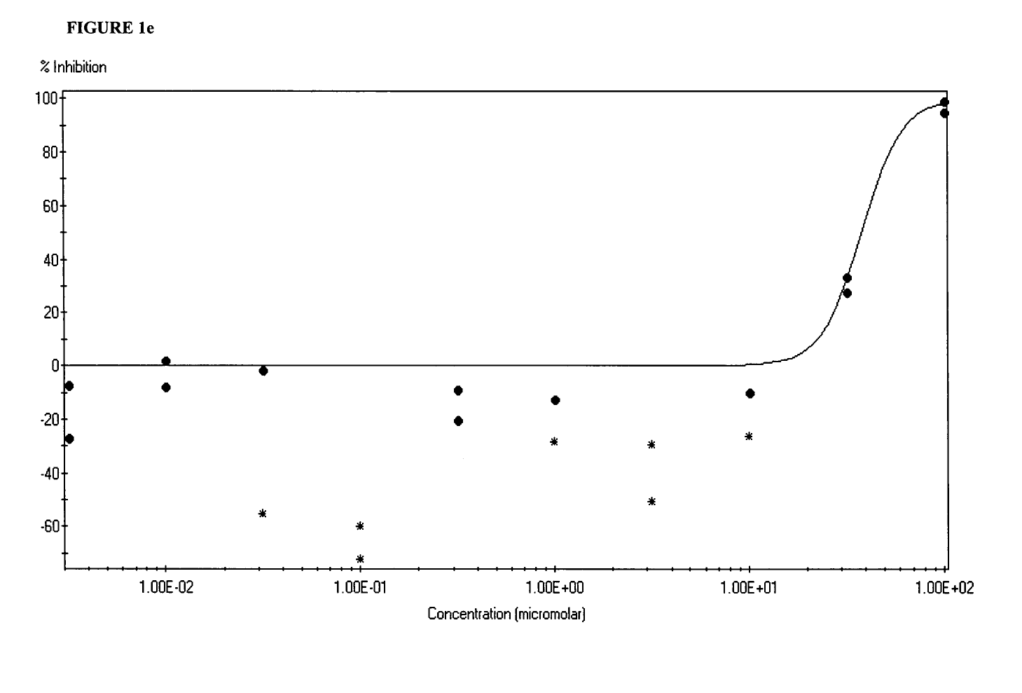

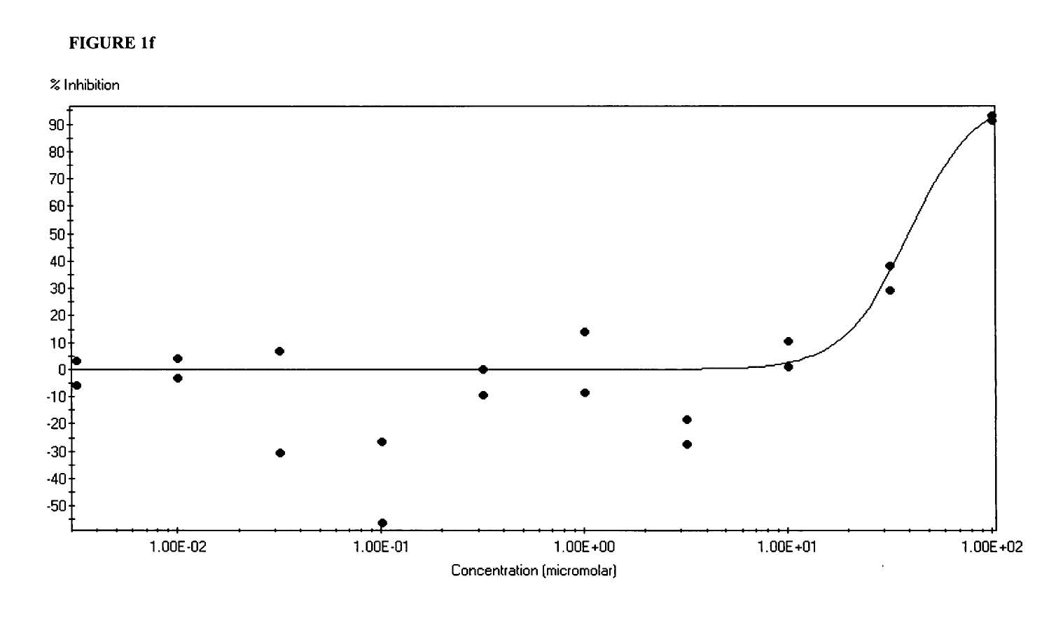

Glycine uptake via the GlyT1 transporter was inhibited by the addition of the thyme extracts or thymol in a dose-dependent manner. Sarcosine, ORG24598 and ALX5407 (all from Sigma, St. Louis, USA) were used as known inhibitors of GlyT1. The measured IC₅₀ values for inhibition of glycine uptake and representative dose-response curves are shown in Table 1 and FIG. 1a-1f, respectively.

FIG. 1 clearly demonstrates that two different thyme extracts as well as the most prominent volatile component of thyme, thymol, can specifically inhibit the action of GlyT1 in a cellular assay.
FIG. 1a shows that for ALX5407, the IC₅₀=6.46 nM
FIG. 1b shows that for Sarcosine, the IC₅₀=35.9 nM
FIG. 1c shows that for ORG 245698, the IC₅₀=0.02 μM
FIG. 1d shows that for Thymol, the IC₅₀=13.6
FIG. 1e shows that for Thyme extract 1, the IC₅₀=48.2 μg/mL
FIG. 1f shows that for Thyme extract 2, the IC₅₀=45.1 μg/mL

Table 1: Measured IC₅₀ values for inhibition of glycine uptake into CHO cells by thyme extract and its volatile components, thymol, p-cymene, linalool, caryophyllene and carvacrol. Data is shown as mean±s.e.m., where the IC₅₀ is stated as μM for pure compounds and as μg/ml for extracts.

TABLE 1

| Substance | IC₅₀ for tritiated glycine uptake |
|---|---|
| Thyme Extract 1 | 48.2 ± 9.8 μg/ml |
| Thyme Extract 2 | 45.1 ± 4.95 μg/ml |
| Thymol (Sigma, Cat. No. | 13.6 ± 0.095 μM |
| T0501) | |
| P-Cymene (Fluka, Cat. No. | inactive |
| T3039) | |
| Linalool (Fluka, Cat. No | inactive |
| 62140) | |
| Caryophyllene (Sigma | inactive |
| Aldrich, Cat. No W225207) | |
| Carvacrol (Sigma, Cat. No | 184 μM |
| W224502) | |

### EXAMPLE 3

### Hippocampal Slice Cultures

Seven-day-old Wistar rats were decapitated using a guillotine. In less than 1 minute the skull was opened, the cerebral hemispheres were separated and transferred and both hippocampi were dissected and transferred into ice cold buffer containing 137 mM NaCl, 5 mM KCl, 0.85 mM Na₂HPO₄, 1.5 mM CaCl₂, 0.66 mM KH₂PO₄, 0.28 mM MgSO₄, 1 mM MgCl₂, 2.7 mM NaHCO₃, 1 mM Kynurenic acid and 0.6% D-glucose.

Transversal hippocampal slices (400 μm) were prepared using a vibrating blade microtome (VT1200S; Leica Microsystems (Schweiz) AG, Heerbrugg, Switzerland) in the same buffer. Hippocampal slices were individually placed on a membrane insert (Millicell Culture Plate Inserts, 0.4 μm) and cultivated at 35° C., 5% CO₂, 95% humidity in a medium containing a 1:1 mixture of BME and MEM (both from Invitrogen) containing 25% heat-inactivated horse serum, 1× GlutaMAX, 1× Penicillin/Streptomycin, 0.6% glucose and 1 mM Kynurenic acid (Stoppini, Buchs and Muller (1991), J. Neurosci. Methods, 37(2), 173-82).

After 48 h in culture, synaptic NMDA receptors were activated by addition of thyme extract or its constituents for 15 min in 140 mM NaCl, 5 mM KCl, 1.3 mM CaCl₂, 25 mM HEPES (pH 7.3), 33 mM D-glucose and 0.02 mM bicuculline methiodide. Sarcosine (100 μM) and ALX5407 (20 nM) were used routinely as positive controls. An additional positive control comprised the addition of 200 μM glycine to sister cultures. After the treatments, sections were washed and fixed for immunohistochemistry. Markers of enhanced synaptic activity, normally associated with long-term potentiation, representing an ex vivo model of learning and memory were quantitated (see Table 2, below).

Table 2. Relative activation of synaptic markers after treatment with thyme extracts or their constituent compounds (thymol, p-cymene, linalool, caryophyllene and carvacrol) in comparison to sister cultures treated with buffer. The activation of any of these markers (or a combination thereof) is observed in classical LTP experiments.

TABLE 2

| Substance | pCREB | pMAPK | GluR1 |
|---|---|---|---|
| Thyme Extract 1 | ± | ++ | 320% |
| Thyme Extract 2 | ± | ++ | 221% |
| Thymol | ++++ | +++ | 250-767% |
| P-Cymene | ++ | ++++ | ± |
| Linalool | ± | ± | ± |
| Caryophyllene | ± | ± | not done |
| Carvacrol | ± | ± | ± |

++++ shows a qualitative maximal activation, whereas ++ signifies a half-maximum activation and ± demonstrated no change in immunoreactivity.

Treatment of hippocampal cultures with thyme extracts as well as with both p-cymene and thymol induced biochemical markers typical for LTP (pCREB: activated form of the cAMP response element binding protein; pMAPK: activated form of the mitogen-activated protein kinase; GLUR1: cell surface presence of AMPA receptor 1).

### EXAMPLE 4

### Effects of Thyme Extract 1 in the Acoustic Startle Response Assay, a Model of Non-Associative Learning and Memory in Zebrafish

Habituation is one of the simplest forms of non-associative learning and memory, resulting in the reduction of a response to a repeated stimulus (Thompson and Spencer (1966), Psychol. Rev., 73 (1), 16-43). One of the prominent behaviours studied in vertebrates is the startle response, a fast contraction of body muscles caused by a sudden acoustic, tactile or visual stimulus mediated by simple neuronal circuitry (Koch (1999), Prog. Neurobiol., 59 (2), 107-128).

Effects of Thyme Extract 1 on acoustic startle response (ASR) were assessed in zebrafish which, at 20 days post fertilization, are known to possess a functional blood-brain-barrier comparable to that of mammals. Test fish were allowed to swim in a 48 well plate (Millipore, Watford, UK), one fish per well, and were exposed to different concentrations of the test compound, as dissolved in their swimming water. 24 h later the fish were placed in an automated live tracking system, which included a Sony XC EI50 CE Camera (Tracksys Ltd., Nottingham, UK) and Ethovision software (Noldus, Wageningen, The Netherlands). After 15 minutes of habituation the fish were exposed to a sequence of auditory tones synchronized by the Ethovision software. Auditory cues of 0.6 second in length, 200 Hz in frequency and 113 decibels, as measured using an NM 102 Noise Meter (NoiseMeter Ltd., Burton Fleming, UK) placed above the 48 well plate, were produced from side-mounted speakers (Bell Packard; placed 10 cm away from the side of the 48 well plate) connected to a Dell computer and given at 1 second intervals (referred to as the inter-trial interval, ITI). An auditory tone session consisted of up to 50 tones, with two sessions being given with 15 minutes recovery period between each episode of auditory stimulation. The ASR was analyzed for each individual fish by measuring the distance moved in response to each auditory stimulus; this provided a quantitative readout of the startle response and was defined as the distance moved by the fish during 1 s from the beginning of the auditory stimulus. Results are shown in Table 3.

TABLE 3
Table 3. In two independent experiments the effects
of Thyme Extract 1 on the ASR were tested.

| Concentration (mg/ml) | Experiment 1 | Experiment 2 |
|---|---|---|
| 0.003 | * | * |
| 0.001 | * | Not Significant |
| 0.0003 | * | Not significant |

Addition of the thyme extract to the fishes' environment was demonstrated to affect their cognitive ability over a large concentration range;
* represents a significant learning difference compared with an age-matched control group exposed to vehicle.

### EXAMPLE 5

### Effects of Thyme Extract in a Traditional Rodent Model of Learning and Memory

Associative learning and memory behavior was also examined in rodents after oral administration of thyme extract, which was identified by the ex vivo LTP assay and proved efficacy in the Zebrafish model. To this aim, mice were subjected to an associative learning and memory paradigm. Mice were individually placed in a reaction box, the floor of which was fitted with a 36V electric grid. When animals receive an electric shock, their normal reaction is to jump up onto an insulated platform to avoid the pain stimulus. The majority of animals that jumped back onto the grid, would, upon receiving the electric shock, rapidly jump back up onto the platform. Animals were trained for 5 min, and the number of times each mouse was shocked, or made an error, was noted. This data constituted the learning data. Re-tests were done at 24 and 48 h, with these trials serving as the memory tests. The number of animals shocked in each group, the time prior to jumping down from the platform and the number of errors in the first 3 min were recorded. After a washout period of five days after conclusion of training, memory decay was tested.

The study included 6 test groups (n=12 per group). Thyme, Ginkgo and vehicle were administered test substances or vehicle via daily oral gavage (10 ml/kg) throughout the study. Treatment dose for Ginkgo biloba was 100 mg/kg BW; thyme extract was tested at 3 doses (40 (low dose), 120 (mid dose), 360 (high dose) mg/kg BW). Rolipram was administered by interaperitoneal injection 30 min before testing (0.1 mg/kg body weight).

When compared to vehicle-treated littermates (negative control) thyme treated animals exhibited a significant better learning and memory performance during the training and memory phase and after the wash-out period and performed as well as mice treated with Gingko biloba or rolipram (positive controls).

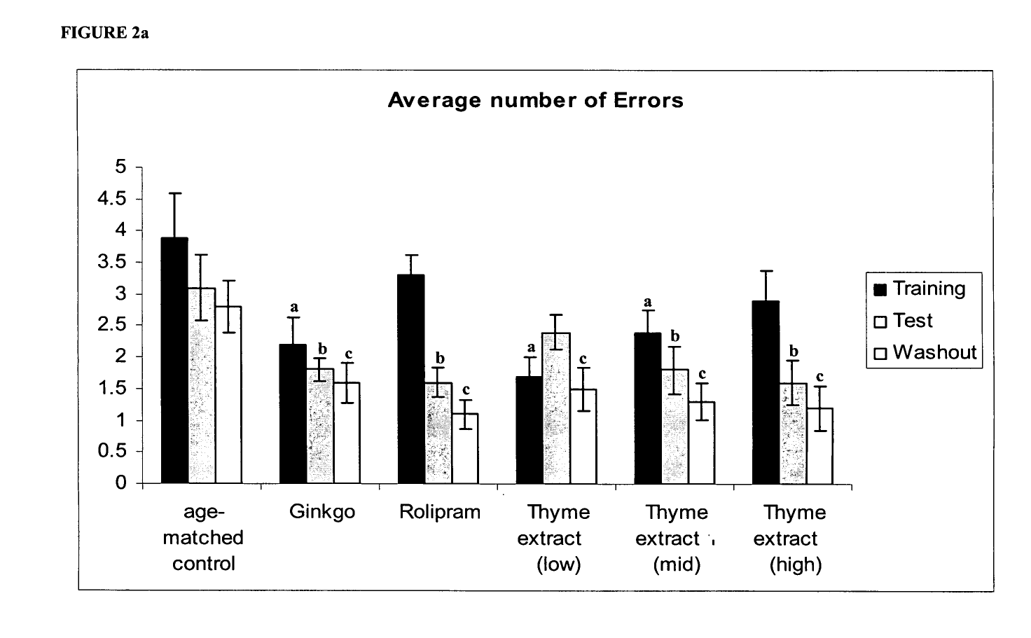

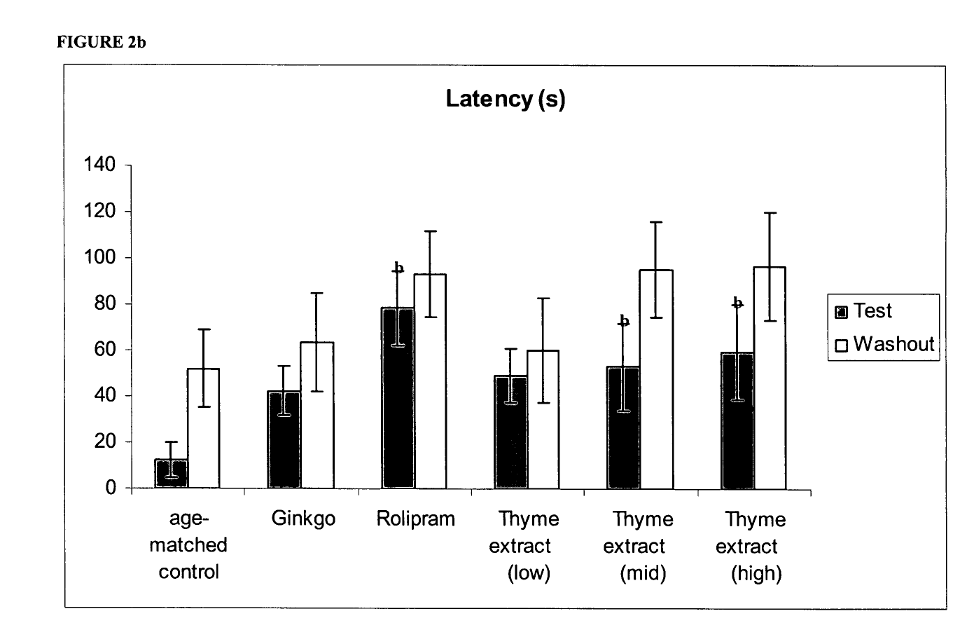

FIG. 2 shows a graph of the step-down behavioral testing results, expressed as the number of errors. As can be seen, mice treated with thyme extract performed significantly better than age-matched controls and comparable to the mice treated with positive control compounds. No statistical difference was observed between the performance of the thyme-treated groups and the ginkgo or rolipram-treated groups at any time. In FIG. 2, “a” indicates a significant difference to vehicle treated age-matched littermates during the training period; “b” indicates a significant difference to vehicle treated age-matched littermates during the test period; and “c” indicates a significant difference to vehicle treated age-matched littermates during the washout period.

### EXAMPLE 6

### Effects of Thyme Extract in New, Totally Automated, Rodent Model of Learning and Memory

We have tested the cognitive performances of mice treated with Ginkgo biloba and thyme extract and compared them with their vehicle treated age-matched controls in the IntelliCage® system (NewBehavior AG, Zurich, Switzerland), which allows automatic monitoring of the animal behaviour over an extended period of time in home cages. IntelliCage® was originally validated for testing experimental animals in cognitive and motivational paradigms (Galsworthy et al. 2005, Behav Brain Res 157: 211-217; Onishchenko et al. 2007, Toxicol Sci 97: 428-437) in social groups without over stress due to social isolation and frequent test environments. Moreover, the IntelliCage® system discriminated rapidly between animals with various degrees of hippocampal damage housed together with controls (Lipp et al. 2004, FENS annual meeting), indicating that the IntelliCage® is suitable for testing hippocampal-dependent behaviour.

### Test Groups and Treatments

The study included 3 test groups (n=12-14 per group; Group 1=vehicle treated mice (control); Group 2=Ginkgo biloba (GBE); Group 3=thyme extract (thyme). All mice were administered test substances or vehicle via daily oral gavage (10 ml/kg) throughout the 8 week study. Treatment dose for Ginkgo biloba was 100 mg/kg BW and for thyme extract 350 mg/kg BW.

### IntelliCages®

The IntelliCage® is a system which enables automated monitoring of spontaneous and learning behaviour of transponder carrying mice in a homecage-like environment (NewBehavior AG, Zurich, Switzerland). Each IntelliCage® is essentially a large rat cage (37.5×55×20.5 cm), into which is placed a metal frame, comprising four recording (operant) chambers. The recording chambers fit into the corners of the cage, each covering a 15×15×21 cm right-angled triangular area of floor space. In-cage antennae enable automatic monitoring of each individual mouse's corner visits; photo-beams within each corner enable automated recording of individual nosepokes and licks of the water bottle spouts. Four triangular mouse shelters were placed in the center of the cage, above which was situated a food hopper, enabling ad libitum access to food.

Each recording chamber contains: (1) a plastic ring (30 mm inner diameter) which serves as an entrance into the chamber and houses the circular antenna which registers corner visits; (2) a grid floor, which the mice sit on once they have entered the chamber; (3) two circular openings (13 mm diameter) which enable access to water bottle spouts; each opening is crossed by photo-beams which register nose-pokes; (4) two motorised doors, which allow (door open) or prohibit (door closed) access to the water bottle spouts; (5) two water bottles; (6) tubing, through which air-puffs can be delivered as aversive stimulation; (7) different coloured light diodes, which can be used for conditioning experiments.

### Experimental Phase:

During a 4-5 day adaptation period mice had free access to all corners, to water and feed and could freely explore the cage. In the next module (nose-poke adaptation, 3 days) mice had to learn to apply nose-pokes. During this phase, all doors were initially closed. Thus, mice had to perform a nose-poke in order to open a door and to reach a water bottle spout. Data collected comprised the same parameters as during the acclimatisation phase; in particular, the least-preferred corner (i.e. that which was visited the least often) of each individual mouse was noted for programming the next modules.

### Object Recognition

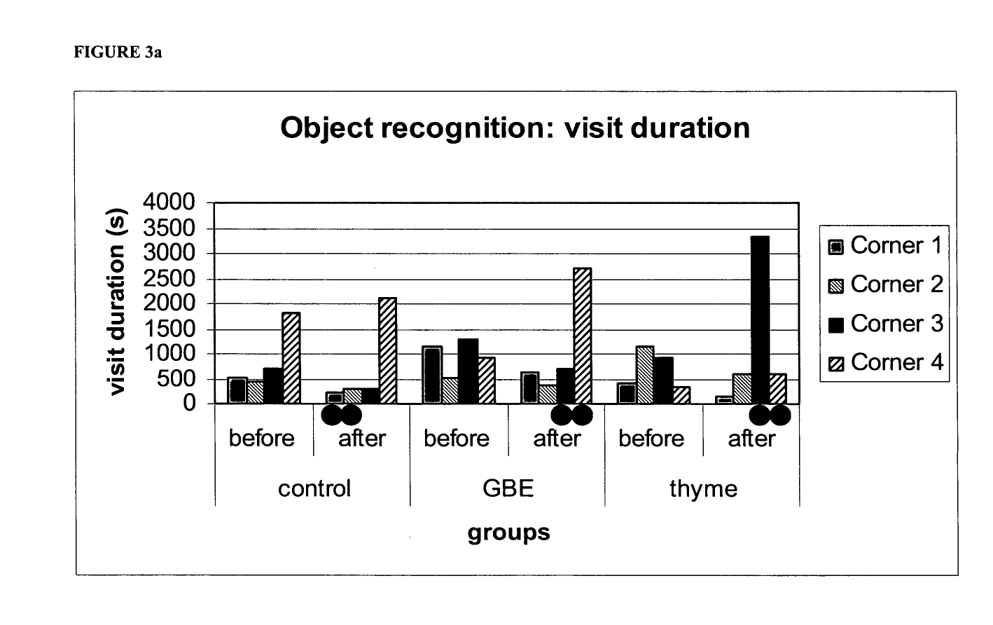

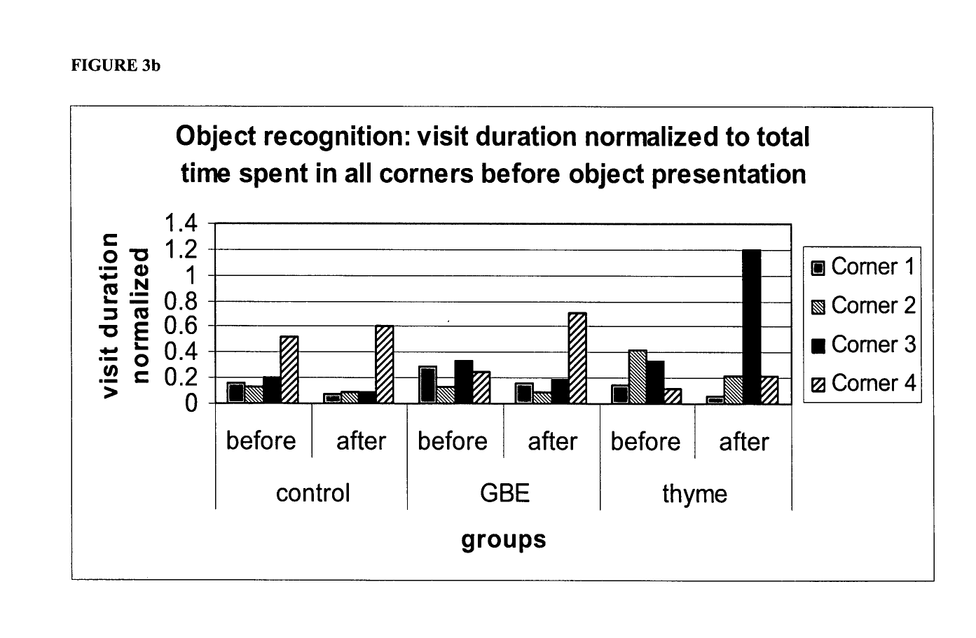

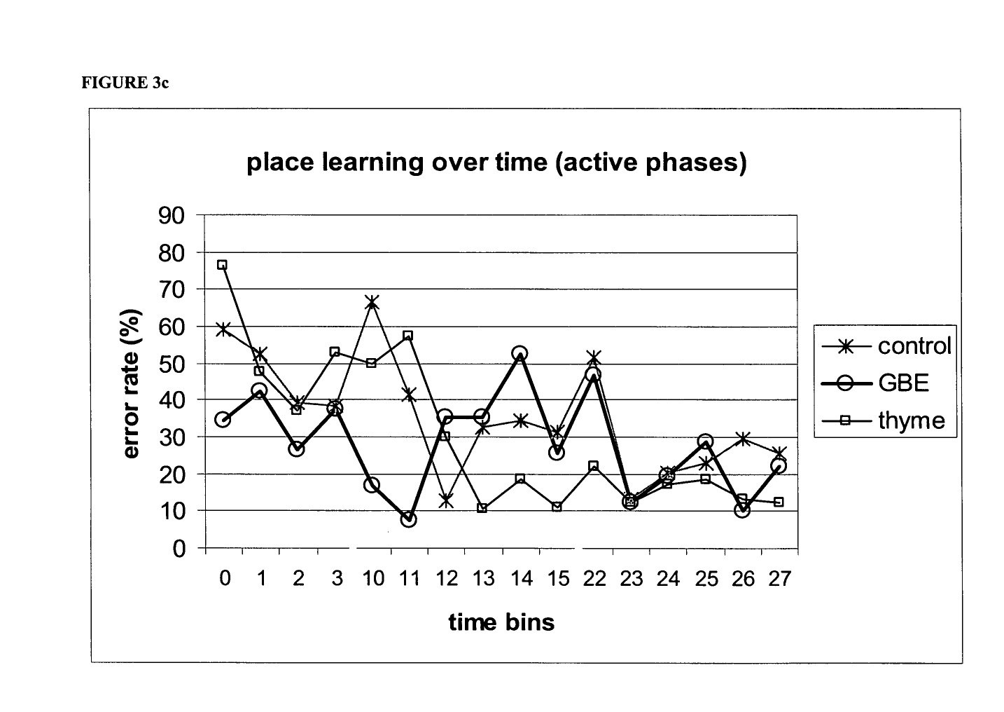

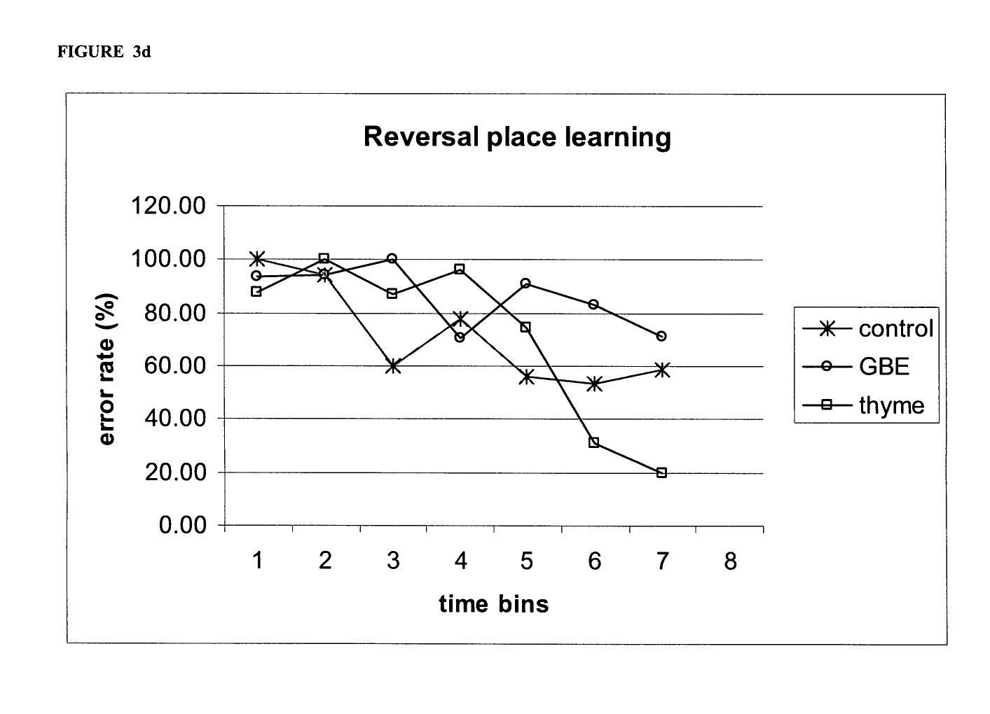

To test the intrinsic exploratory activity of the mice, two identical objects were placed in either corners 1 and 2 or in corners 3 and 4 respectively. The animals had the opportunity to explore the cage and had free access to water and feed. Visits were recorded 3 h before and 3 h after the objects were presented. FIG. 3a depicts the results from an object recognition test. For the Ginkgo group, a significant increase was seen for the visiting time in corner 4. For thyme extract a clear increase in visiting time was seen in corner 3.

FIG. 3a: Visit duration in each corner 3 h before and after objects were presented. The filled circles represent the place of an object. p=0.001 for thyme extract in corner 3, p=0.004 for Ginkgo biloba in corner 4, respectively.

### Place Learning (Measure of Learning Capacity)

In order to investigate place learning behavior, mice were tested in this module. The least-preferred corner, as determined during the nose-poke adaptation phase, was designated as the “correct” corner for each individual mouse; only nose-pokes within this corner would trigger opening of the motorised doors and permit access to the water bottles; nose-pokes in all other corners were “incorrect” and resulted in aversive stimulation, in the form of an air puff (1 s).

FIG. 3b shows the learning curve during the active phase for the thyme extract treated group in comparison with control and Ginkgo biloba (GBE)-treated animals. All groups performed better over time, although no difference among the control and treatment groups could be found (p=0.44). This data shows that all animals successfully learned the task.

### Reversal of Place Learning

In this module, the “correct” corner was designated as that which was diagonally opposite to the “correct” corner of the previous test module. Visits to “incorrect” corners were again subjected to negative reinforcement (an air-puff). As expected, the initial error rate was high at the beginning of this module, but all groups quickly learned the task. There was no difference between the groups during the first 10 h. Moreover, the thyme extract-treated group performed significantly better than both other groups by the end of the test period.

FIG. 3c: Reversal of place learning: Errors were recorded during the first active phase. Error rates declined from 100% to about 20% for thyme extract and about 60-70% for both control group and the GBE-treated, suggesting that thyme-treated animals performed better that ginkgo-treated mice in this behavioral test (p=0.011 for the last two time bins). Time bins correspond to 2 h each.

### EXAMPLE 7

### Preparation of a Soft Gelatine Capsule

A soft gelatine capsule may be prepared comprising the following ingredients:

| Ingredient | Amount per Capsule |
|---|---|
| Enriched thyme extract | 200 mg |
| Lecithin | 50 mg |
| Soy bean oil | 250 mg |

Two capsules per day for 3 months may be administered to a human adult. Cognitive functions, alertness and the ability to focus on work are seen to improve.

### EXAMPLE 8

### Preparation of an Instant Flavoured Soft Drink

| Ingredient | Amount [g] |
|---|---|
| Enriched thyme extract | 0.9 |
| Sucrose, fine powder | 922.7 |
| Ascorbic acid, fine powder | 2.0 |
| Citric acid anhydrous powder | 55.0 |
| Lemon flavour | 8.0 |
| Tri sodium citrate anhydrous powder | 6.0 |
| Tricalciumphosphate | 5.0 |
| β-Carotene 1% CWS from DNP AG, | 0.4 |
| Kaiseraugst, Switzerland | |
| Total amount | 1000 |

All ingredients are blended and sieved through a 500 μm sieve. The resulting powder is put in an appropriate container and mixed in a tubular blender for at least 20 minutes. For preparing the drink, 125 g of the obtained mixed powder are mixed with sufficient water to produce one litre of beverage.

The ready-to-drink soft drink contains ca. 30 mg enriched thyme extract per serving (250 ml). As a strengthener and for general well-being 2 servings per day (500 ml) may be drunk.

### EXAMPLE 9

### Dry Dog Feed Containing Thymol and/or P-Cymene or Thyme Extract

A commercial basal diet for dogs (e.g. Mera Dog “Brocken”, MERA-Tiernahrung GmbH, Marienstraβe 80-84, D-47625 Kevelaer-Wetten, Germany) is sprayed with a suspension of corn oil containing thymol and/or p-cymene or thyme extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a dog a daily dose of 50 mg thymol and/or p-cymene or thyme extract per kg body weight. The food composition is dried to contain dry matter of about 90% by weight. For an average dog of 10 kg body weight to consume approx. 200 g dry feed per day, the dog food contains approx. 2500 mg thymol and/or p-cymene or thyme extract per kg food. For heavier dogs, the feed mix is prepared accordingly. For reduction of stress, fear and aggressiveness in dogs, the food can be given to dogs in animal shelter farms on a regular basis. Before veterinarian visits or stays in veterinarian clinics or holiday separation, the food is given at least one week before, during the stressful event and one week thereafter.

### EXAMPLE 10

### Wet Cat Food Containing Thymol and/or P-Cymene or Thyme Extract

A commercial basal diet for cats (e.g. Happy Cat “Adult”, Tierfeinnahrung, Südliche Hauptstraβe 38, D-86517 Wehringen, Germany) is mixed with a suspension of corn oil containing thymol and/or p-cymene or thyme extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a cat a daily dose of 100 mg thymol and/or p-cymene or thyme extract per kg body weight. For an average cat of 5 kg of body weight to consume approx. 400 g of wet food, the cat food contains 1250 mg thymol and/or p-cymene or thyme extract per kg food. The food composition is dried to contain dry matter of about 90% by weight. For reduction of stress, fear and aggressiveness in cats, the food can be given to cats in animal shelter farms on a regular basis. Before veterinarian visits or stays in veterinarian clinics, the food is given at least one week before, during the stressful event and one week thereafter.

### EXAMPLE 11

Dog Treats Containing Thymol and/or P-Cymene or Thyme Extract

Commercial dog treats (e.g. Mera Dog “Biscuit” for dogs as supplied by Mera Tiernahrung GmbH, Marienstrasse 80-84, 47625 Kevelaer-Wetten, Germany) are sprayed with a suspension of corn oil containing thymol and/or p-cymene or thyme extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to the treats 5-50 mg thymol and/or p-cymene or thyme extract per g treats. The food composition is dried to contain dry matter of about 90% by weight. To reduce fear and tension, the treat can be given during the day in addition to the food, or when feeding is not warranted, i.e. upon travels, for up to 5 times per day.

### EXAMPLE 12

### Cat Treats Containing Thymol and/or P-Cymene or Thyme Extract

Commercial cat treats (e.g. Whiskas Dentabits for cats as supplied by Whiskas, Masterfoods GmbH, Eitzer Str. 215, 27283 Verden/Aller, Germany) are sprayed with a suspension of corn oil containing thymol and/or p-cymene or thyme extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to the treats 5-50 mg thymol and/or p-cymene or thyme extract per g treats. The food composition is dried to contain dry matter of about 90% by weight. To reduce fear and tension, the treat can be given during the day in addition to the food, or when feeding is not warranted, i.e. upon travels, for up to 5 times per day.

What is claimed is:

1. A method of improving cognitive function and/or psychosocial status in an animal or human comprising administering a cognitive function-improving or psychosocial status improving amount of thymol and/or p-cymene.

2. A method according to claim 1 comprising administering thymol.

3. A method according to claim 2 wherein the thymol is a component of a plant extract.

4. A method according to claim 2 wherein the plant extract is a Thymus extract.

5. A method according to claim 4 where in the Thymus extract contains at least about 25-80% thymol.

6. A method according to claim 1 comprising administering p-cymene.

7. A method according to claim 6 wherein p-cymene is a component of a plant extract.

8. A method according to claim 7 wherein the plant extract is a Thymus extract.

9. A method according to claim 8 wherein the thyme extract contains as least about 5-55% p-cymene.

10. A method according to claim 1 wherein the cognitive function and/or psychological status is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilization of mental status, a stress reliever, work-overload stress, stress-related exhaustion and/or burn out, and to promote relaxation.

11. A composition used in the manufacture of a nutraceutical or medicament for improving cognitive function or psycho-social status in an animal or human, which comprises a cognitive function-improving amount or psychosocial status improving amount of thymol and/or p-cymene.

12. A composition according to claim 11 comprising thymol.

13. A composition according to claim 12 wherein the thymol is a component of a plant extract.

14. A composition according to claim 13 which is a Thymus extract.

15. A composition according to claim 14 wherein the Thymus extract contains at least about 25-80% thymol.

16. A composition according to claim 11, wherein the composition comprises p-cymene.

17. A composition according to claim 16, wherein the p-cymene is a component of a plant extract.

18. A composition according to claim 16, which is a Thymus extract.

19. A composition according to claim 18 wherein the Thymus extract contains at least about 5-55% p-cymene.

20. A composition according to claim 11 wherein the cognitive function or psycho social status is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilization of mental status, a stress reliever, work-overload stress, stress-related exhaustion and/or burn out, and to promote relaxation.
